(19) United States
(12) Patent Application Publication
(10) Pub. No.: US 2026/0158066 A1
(43) Pub. Date: Jun. 11, 2026

(54) Cell Therapeutics With Improved Persistence In Vivo And Antitumor Effect, And A Method For Producing Thereof

(71) Applicant: Korea Advanced Institute of Science and Technology, Daejeon (KR)

(72) Inventors: Heung Kyu LEE, Daejeon (KR); Won Hyung PARK, Daejeon (KR)

(21) Appl. No.: 19/351,416

(22) Filed: Oct. 7, 2025

(30) Foreign Application Priority Data
Dec. 9, 2024 (KR) 10-2024-0181651

(51) Int. Cl. A61K 35/17; A61K 45/06; A61P 35/00

(52) U.S. Cl. CPC A61K 35/17; A61K 45/06; A61P 35/00

(57) ABSTRACT

The present invention relates to a cell therapeutic with improved proliferation rate, in vivo persistence, and antitumor effect, and a method for producing the same. Conventional γδ cell therapeutics have failed to achieve significant therapeutic effects due to their inability to overcome immune suppression, resulting in short in vivo persistence and reduced antitumor immune responses. However, γδ T cells produced by the special culturing method or genetic modification of the present invention show remarkably improved proliferation rate, in vivo persistence, and antitumor effects, and are expected to significantly improve the survival rate of cancer subjects when used alone or in combination with conventional cancer immunotherapies.

### TECHNICAL FIELD

The present invention relates to a γδ cell therapeutic with improved proliferation rate, in vivo persistence, and antitumor effect, and a method for producing the same.

### BACKGROUND ART

Cancer is one of the most common causes of death worldwide. Approximately ten million new cases occur each year, accounting for about 12% of all causes of death, making it the third leading cause of death. Accordingly, efforts have been continuously made to develop effective anticancer agents; however, brain cancer, particularly glioblastoma, still has a survival rate of less than 10%, and the development of effective therapeutics remains extremely insufficient. Even cancer immunotherapy—which claims to have opened a new horizon in cancer treatment by utilizing the body's immune system to attack cancer cells—fails to exert proper effects in glioblastoma. This is because immunosuppressive mechanisms that suppress immune cells are highly active in brain cancer, and due to the nature of the brain as an organ, the existence of the blood-brain barrier makes treatment and immune system activation difficult. In fact, a representative immune checkpoint blockade, PD-1 inhibitor, recently failed in a phase 3 clinical trial for brain cancer treatment, and development of CAR T-cell therapeutics is also facing difficulties.

Meanwhile, γδ T cells are important cells associated with good prognosis in brain tumor patients, and unlike existing personalized cell therapeutics, they have the great advantage of being suitable for allogeneic transplantation. Hence, their application as off-the-shelf cell therapeutics for anticancer use has been attempted. However, conventional γδ T-cell therapeutics have failed to overcome immune suppression and have shown poor therapeutic effects due to short in vivo persistence and decreased antitumor immune responses. Therefore, in order to actively utilize γδ T cells as a treatment for intractable brain cancer, there has been a need to enhance their in vivo persistence and antitumor immune responses.

The present invention was devised to solve the above problems and relates to a γδ T cell therapeutic with improved proliferation rate, in vivo persistence, and antitumor effect, and a method for producing the same. The inventors of the present invention have succeeded in producing γδ T cells with significantly improved proliferation rate, in vivo persistence, and antitumor effect by culturing γδ T cells using a special method or genetically engineering them. Since the γδ T cells of the present invention are expected to significantly improve the survival rate of cancer subjects when used alone or in combination with conventional cancer immunotherapies, they can be widely utilized in the healthcare and medical fields.

### DISCLOSURE

### Technical Problem

One object of the present invention is to provide a cell therapeutic for the prevention or treatment of cancer.

Another object of the present invention is to provide a method for producing the cell therapeutic.

Yet another object of the present invention is to provide a pharmaceutical composition for the prevention or treatment of cancer comprising the cell therapeutic.

The cell therapeutic of the present invention is characterized by increased expression of Batf3 (Basic Leucine Zipper ATF-Like Transcription Factor 3), resulting in enhanced proliferation rate, increased in vivo persistence, or improved antitumor effect.

However, the technical problems to be solved by the present invention are not limited to those mentioned above, and other problems not mentioned will be apparent to those skilled in the art from the following description.

### Technical Solution

Hereinafter, various embodiments described herein will be described with reference to figures. In the following description, numerous specific details are set forth, such as specific configurations, compositions, and processes, etc., in order to provide a thorough understanding of the present disclosure. However, certain embodiments may be practiced without one or more of these specific details, or in combination with other known methods and configurations. In other instances, known processes and preparation techniques have not been described in particular detail in order to not unnecessarily obscure the present disclosure. Reference throughout this specification to “one embodiment” or “an embodiment” means that a particular feature, configuration, composition, or characteristic described in connection with the embodiment is included in at least one embodiment of the present disclosure. Thus, the appearances of the phrase “in one embodiment” or “an embodiment” in various places throughout this specification are not necessarily referring to the same embodiment of the present disclosure. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present disclosure pertains.

In the present invention, the term “γδ T cell” refers to a type of T cell expressing a gamma-delta (γδ) T cell receptor (T cell antigen receptor; TCR). T cells, which are a type of lymphocyte, constitute the main axis of adaptive immunity together with B cells. T cells are generated through a process in which progenitors originating from hematopoietic stem cells mature in the thymus. T cells consist of the majority αβ T cells, which express an alpha-beta (αβ) T cell receptor circulating between the thymus and peripheral lymphoid tissue, and the minority γδ T cells, which express a gamma-delta (γδ) T cell receptor. γδ T cells are the earliest T cells to migrate out of the thymus and most of them relocate prenatally to the skin, intestine, lung, and epithelial tissues. Although γδ T cells do not have extensive TCR diversity, they respond to a variety of biological injuries inflicted on epithelial tissues, although little is known about the self-antigens that induce such responses. γδ T cells account for only approximately 0.5-5% of human peripheral blood T cells and functionally participate in both adaptive immunity and innate immunity. Human γδ T cells exist as two major subsets according to their TCR chain composition: the Vδ1 chain or the Vδ2 chain. Most of the γδ T cells in the blood carry the Vδ2 chain and exist as a pair with the Vγ9 chain. γδ T cells arise earlier than αβ T cells in the thymus and possess limited TCR diversity for several antigens, including phosphoantigens, but they induce an innate-like immune response by reacting faster than αβ T cells. γδ T cells, like αβ T cells, have a stronger antitumor effect through cytolysis, but unlike αβ T cells—which are the main cause of graft-versus-host disease (GVHD) because they react to cells expressing non-self MHC—γδ T cells are not MHC-restricted and are known not to induce GVHD. This suggests that γδ T cells may be a promising off-the-shelf cell therapeutic for cancer immunotherapy. Accordingly, various techniques have been devised to secure a sufficient number of γδ T cells as an off-the-shelf cell therapeutic. For example, it has been found that treatment with interleukin-2 (IL-2), interleukin-5 (IL-5), zoledronic acid (ZA, ZOL), phytohaemagglutinin (PHA), transforming growth factor beta (TGF-β), concanavalin A, or amphotericin B specifically proliferates γδ T cells. Based on this finding, many preclinical and clinical trials have attempted to apply γδ T cells obtained by the above methods as therapeutics for various cancers. However, it has been confirmed that γδ T cells expanded in vitro survive only a few days after transplantation into an individual. Thus, it remains a technical challenge to maintain the in vivo persistence of γδ T cells and to further improve their proliferation rate in vivo.

Therefore, the present invention provides a γδ T cell therapeutic whose proliferation rate, in vivo persistence, and antitumor effect are remarkably enhanced by increasing the expression of Batf3 (Basic Leucine Zipper ATF-Like Transcription Factor 3) in γδ T cells as a solution to the above-mentioned technical problems.

In the present invention, the term “cell therapeutic” refers to a pharmaceutical product (according to U.S. FDA regulations) consisting of cells and tissues that are isolated, cultured, and specially manipulated from an individual and used for therapeutic, diagnostic, or preventive purposes. This term encompasses a series of actions such as proliferating/selecting living autologous, allogeneic, or xenogeneic cells ex vivo, or otherwise altering the biological characteristics of cells, in order to restore the function of cells and tissues, and the resulting product is used for therapeutic, diagnostic, or preventive purposes. The United States has regulated cell therapeutics as pharmaceuticals since 1993, and Korea has done so since 2002. Such cell therapeutics can be broadly classified into two categories: the first is stem cell therapeutics for tissue regeneration or organ function recovery, and the second is immune cell therapeutics for modulating immune responses, such as suppression or enhancement of immune reactions in vivo. In the present invention, the cell therapeutic is specifically an immune cell therapeutic derived from γδ T cells, and more specifically, it is characterized by γδ T cells with increased expression of Batf3 (Basic Leucine Zipper ATF-Like Transcription Factor 3). The cell therapeutic of the present invention can be usefully applied to the treatment of cancer, particularly brain cancer, and is expected to produce a synergistic effect when used in combination with conventional anticancer agents, immunotherapeutic agents, or immune checkpoint inhibitors.

In the present invention, the term “brain cancer” is used interchangeably with “brain tumor,” referring to tumors that develop within the skull, including tumors in the brain and spinal canal, and is a general term for tumors arising in the brain parenchyma or meninges. This term encompasses neoplasms that grow inside the skull, including those originating from brain cells, blood vessels, nerves, meninges, and other structures. Brain cancer has characteristics distinct from those of ordinary cancers, owing to the unique structure of the brain compared to other organs and tissues. Specifically, the blood vessels of the brain have a tight boundary called the blood-brain barrier (BBB), which prevents tumor cells from easily metastasizing to other organs via the bloodstream even when tumors arise in the brain. This is also the reason why anticancer drugs are poorly delivered to brain tumors. In addition, unlike other cancers that are generally classified by stage, brain cancers are graded. The grading is based on factors such as the rate of tumor cell division: grade 1 is generally benign, grade 2 is borderline, and grades 3-4 are malignant. However, glioblastoma can be clinically classified as malignant even if it is grade 1 or 2. The brain cancer referred to in the present invention may be at least one selected from the group consisting of glioblastoma, malignant glioma, lymphoma, germinoma, and metastatic tumors, but is not limited thereto.

In the present invention, the term “immune checkpoint inhibitor” refers to a drug that activates T cells to attack cancer cells by blocking the activity of immune checkpoint proteins involved in suppressing T cells. That is, cancer cells avoid immune cell attack by expressing self-tolerance proteins that make them recognized as self, thereby escaping immune surveillance. These drugs inhibit immune evasion signals so that immune cells attack and eliminate cancer cells. Representative novel drugs developed to neutralize this immune evasion mechanism of cancer cells include substances that specifically bind to proteins such as CTLA-4, PD-1, PD-L1, PD-L2, LAG-3, TIM-3, BTLA, or KIR. Here, the term “substance” is used in a broad sense including antibodies and is not limited thereto. In the present invention, the immune checkpoint inhibitor may comprise at least one selected from the group consisting of CTLA-4 receptor inhibitors, PD-1 receptor inhibitors, PD-L1 ligand inhibitors, PD-L2 ligand inhibitors, LAG-3 receptor inhibitors, TIM-3 receptor inhibitors, BTLA receptor inhibitors, and KIR receptor inhibitors. Immune checkpoint inhibitors are fourth-generation anticancer drugs that utilize the immune system of the cancer-bearing individual, and successful treatment results have been reported in various cancer types. However, they have consistently failed to show significant effects in brain cancers. Several clinical trials have attempted to introduce immune checkpoint inhibitors for glioblastoma treatment in recent years, but none have yielded results significantly superior to conventional therapies. Thus, treatment of brain tumors using immune checkpoint inhibitors still requires additional breakthroughs.

In the present invention, the term “prevention” may include, without limitation, all acts of inhibiting the occurrence of cancer itself, blocking symptoms caused by cancer, or suppressing or delaying such symptoms, by using a composition comprising the active ingredient of the present invention.

In the present invention, the terms “treatment” or “improvement” may include, without limitation, all acts in which symptoms caused by cancer occurrence or invasion are alleviated or improved by using a composition comprising the active ingredient of the present invention.

In the present invention, the pharmaceutical composition, unlike conventional anticancer agents that directly affect cancer cells, is a therapeutic agent that enhances the body's natural immunity by activating the immune system, thereby enabling the body's own immune cells to attack cancer cells. For the purpose of the present invention, the pharmaceutical composition refers to a therapeutic agent capable of improving the function and activity of T cells present in the cancer-bearing individual. The pharmaceutical composition of the present invention may be used in combination with conventional anticancer agents other than immune checkpoint inhibitors or with immunotherapeutics. In this case, the conventional anticancer agent is a chemical anticancer agent and may include at least one selected from the group consisting of: nitrogen mustard, imatinib, oxaliplatin, rituximab, erlotinib, neratinib, lapatinib, gefitinib, vandetanib, nilotinib, semaxanib, bosutinib, axitinib, cediranib, lestaurtinib, trastuzumab, gefitinib, bortezomib, sunitinib, carboplatin, sorafenib, bevacizumab, cisplatin, cetuximab, viscum album, asparaginase, tretinoin, hydroxycarbamide, dasatinib, estramustine, gemtuzumab ozogamicin, ibritumomab tiuxetan, heptaplatin, methyl aminolevulinate, amsacrine, alemtuzumab, procarbazine, alprostadil, holmium nitrate chitosan, gemcitabine, doxifluridine, pemetrexed, tegafur, capecitabine, gimeracil, oteracil, azacitidine, methotrexate, uracil, cytarabine, fluorouracil, fludarabine, enocitabine, flutamide, capecitabine, decitabine, mercaptopurine, thioguanine, cladribine, carmofur, raltitrexed, docetaxel, paclitaxel, irinotecan, belotecan, topotecan, vinorelbine, etoposide, vinblastine, idarubicin, mitomycin, bleomycin, dactinomycin, pirarubicin, aclarubicin, peplomycin, temsirolimus, temozolomide, busulfan, ifosfamide, cyclophosphamide, melphalan, altretamine, dacarbazine, thiotepa, nimustine, chlorambucil, mitolactol, leucovorin, tretinoin, exemestane, aminoglutethimide, anagrelide, olaparib, vinorelbine tartrate, fadrozole, tamoxifen, toremifene, testolactone, anastrozole, letrozole, vorozole, bicalutamide, lomustine, 5-FU, vorinostat, entinostat, and carmustine. The immunotherapeutic agent may be at least one selected from the group consisting of pembrolizumab, ipilimumab, nivolumab, atezolizumab, and durvalumab, but is not limited thereto.

The pharmaceutical composition of the present invention may be formulated in the form of an injectable preparation, capsule, tablet, granule, powder, or beverage, and may be administered to humans.

Preferably, the composition is formulated as an injection, but it can also be formulated by conventional methods as an oral dosage form such as a powder, granule, capsule, tablet, aqueous suspension, or as a topical agent, suppository, or sterile injectable solution. The pharmaceutical composition may further include a pharmaceutically acceptable carrier. When administered orally, the pharmaceutically acceptable carrier may include binders, excipients, disintegrants, solubilizers, dispersing agents, stabilizers, suspending agents, colorants, and flavoring agents. For injection, buffers, preservatives, analgesics, solubilizers, isotonic agents, and stabilizers may be used. For topical administration, bases, excipients, lubricants, and preservatives may be used. Examples of carriers, excipients, and diluents suitable for the formulation of the present invention include lactose, dextrose, sucrose, sorbitol, mannitol, xylitol, erythritol, maltitol, starch, gum acacia, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate, or mineral oil. In addition, fillers, anti-caking agents, lubricants, wetting agents, flavoring agents, emulsifiers, and preservatives may be additionally included.

When the pharmaceutical composition of the present invention is used for therapeutic purposes, various factors such as age, weight, general health, sex, diet, administration time, administration route, excretion rate, drug combination, and severity of the specific disease to be prevented or treated in the subject can be considered, and the dosage can vary accordingly. The dosage of the pharmaceutical composition is appropriately selected by those skilled in the art depending on the condition of the patient, body weight, severity of the disease, dosage form, route and duration of administration. Administration may be performed once daily or divided into several doses. The dosage does not limit the scope of the present invention in any respect.

The cell therapeutic included in the pharmaceutical composition of the present invention is intended to enhance anticancer activity and may be administered by any device that allows it to migrate to a target cell or tissue, and may be included in a therapeutically effective amount for treating the target disease. The term “therapeutically effective amount” means the amount of an active ingredient or pharmaceutical composition that elicits a biological or medical response in a tissue system, animal, or human that is deemed desirable by a researcher, veterinarian, physician, or other clinician, including an amount that induces alleviation of symptoms of the disease or disorder being treated. It will be apparent to those skilled in the art that the amount of the cell therapeutic included in the composition may vary depending on the desired effect. Thus, the optimal content of the cell therapeutic can be easily determined by those skilled in the art and may be adjusted depending on factors such as the type of disease, severity of the disease, content of other ingredients contained in the composition, type of dosage form, and the patient's age, weight, general health, sex, diet, administration time, administration route, secretion rate of the composition, treatment period, and concomitant drugs, among others. Taking all of these factors into consideration, the composition may include an amount that achieves the maximum effect with the minimum dose without causing side effects.

Hereinafter, in one embodiment of the present invention, there is provided a method for producing a γδ T cell therapeutic with increased proliferation rate or in vivo persistence, the method comprising the step of stimulating the γδ TCR (T cell antigen receptor) of γδ T cells isolated from a subject. The production method of the present invention may further comprise, following the step of stimulating the γδ TCR of γδ T cells isolated from the subject, the step of restimulating the γδ TCR of the γδ T cells; or the step of overexpressing Batf3 (Basic Leucine Zipper ATF-Like Transcription Factor 3). In this case, the stimulation or restimulation of the γδ TCR may be performed by treating the cells with a composition comprising an anti-γδ TCR antibody, and the composition comprising the anti-γδ TCR antibody may further include one or more selected from the group consisting of anti-CD28 antibody, Interleukin-2 (IL-2), Interleukin-5 (IL-5), Zoledronic acid, PHA (phytohaemagglutinin), Transforming growth factor beta, Concanavalin A, and Amphotericin B. The step of restimulating the γδ TCR of the γδ T cells may be performed 3 to 7 days, 4 to 6 days, 3 to 6 days, 4 to 7 days, or 4.5 to 5.5 days after the step of stimulating the γδ TCR of the γδ T cells isolated from the subject, preferably after 5 days, but is not limited thereto. In addition, the step of overexpressing Batf3 (Basic Leucine Zipper ATF-Like Transcription Factor 3) may be performed 3 to 72 hours, 3 to 60 hours, 3 to 48 hours, 3 to 24 hours, 6 to 72 hours, 12 to 72 hours, 24 to 72 hours, 48 to 72 hours, 6 to 60 hours, 9 to 48 hours, 12 to 36 hours, or 18 to 30 hours after the step of stimulating the γδ TCR of the γδ T cells, and preferably 20 to 28 hours thereafter, but is not limited thereto. Further, in the production method of the present invention, the step of overexpressing Batf3 may be carried out by transducing a vector comprising an PGK promoter (phosphoglycerate kinase 1 promoter) and the Batf3 gene, but is not limited thereto, and it will be apparent to those skilled in the art that any known method for overexpressing Batf3 may be used without limitation.

In another embodiment of the present invention, there is provided a method for improving the proliferation rate or in vivo persistence of γδ T cells, the method comprising the step of stimulating the γδ TCR (T cell antigen receptor) of γδ T cells isolated from a subject. The method of the present invention may further comprise, following the step of stimulating the γδ TCR of γδ T cells isolated from the subject, the step of restimulating the γδ TCR of the γδ T cells; or the step of overexpressing Batf3 (Basic Leucine Zipper ATF-Like Transcription Factor 3). In this case, the stimulation or restimulation of the γδ TCR may be performed by treating the cells with a composition comprising an anti-γδ TCR antibody, and the composition comprising the anti-γδ TCR antibody may further include one or more selected from the group consisting of anti-CD28 antibody, Interleukin-2 (IL-2), Interleukin-5 (IL-5), Zoledronic acid, PHA (phytohaemagglutinin), Transforming growth factor beta, Concanavalin A, and Amphotericin B. The step of restimulating the γδ TCR of the γδ T cells may be performed 3 to 7 days, 4 to 6 days, 3 to 6 days, 4 to 7 days, or 4.5 to 5.5 days after the step of stimulating the y 8 TCR of the y 8 T cells isolated from the subject, preferably after 5 days, but is not limited thereto. In addition, the step of overexpressing Batf3 (Basic Leucine Zipper ATF-Like Transcription Factor 3) may be performed 3 to 72 hours, 3 to 60 hours, 3 to 48 hours, 3 to 24 hours, 6 to 72 hours, 12 to 72 hours, 24 to 72 hours, 48 to 72 hours, 6 to 60 hours, 9 to 48 hours, 12to 36 hours, or 18 to 30 hours after the step of stimulating the γδ TCR of the γδ T cells, and preferably 20 to 28 hours thereafter, but is not limited thereto. Further, in the method of the present invention, the step of overexpressing Batf3 may be carried out by transducing a vector comprising an PGK promoter (phosphoglycerate kinase 1 promoter) and the Batf3 gene, but is not limited thereto, and it will be apparent to those skilled in the art that any known method for overexpressing Batf3 may be used without limitation.

In yet another embodiment of the present invention, there is provided a γδ T cell therapeutic produced by the above-described method for producing a γδ T cell therapeutic with increased proliferation rate or in vivo persistence. The γδ T cell therapeutic may be used for the treatment of cancer, wherein the cancer may be at least one selected from the group consisting of brain cancer, breast cancer, cervical cancer, melanoma, lung cancer, bladder cancer, prostate cancer, leukemia, kidney cancer, liver cancer, colon cancer, pancreatic cancer, gastric cancer, gallbladder cancer, ovarian cancer, lymphoma, osteosarcoma, uterine cancer, oral cancer, bronchial cancer, nasopharyngeal cancer, laryngeal cancer, skin cancer, hematological cancer, thyroid cancer, parathyroid cancer, ureter cancer, adenocarcinoma, and thymic cancer, preferably brain cancer, but not limited thereto.

In yet another embodiment of the present invention, there is provided a pharmaceutical composition for the prevention or treatment of cancer, comprising, as an active ingredient, a γδ T cell therapeutic produced by the above-described method for producing a γδ T cell therapeutic with increased proliferation rate or in vivo persistence. The cancer may be at least one selected from the group consisting of brain cancer, breast cancer, cervical cancer, melanoma, lung cancer, bladder cancer, prostate cancer, leukemia, kidney cancer, liver cancer, colon cancer, pancreatic cancer, gastric cancer, gallbladder cancer, ovarian cancer, lymphoma, osteosarcoma, uterine cancer, oral cancer, bronchial cancer, nasopharyngeal cancer, laryngeal cancer, skin cancer, hematological cancer, thyroid cancer, parathyroid cancer, ureter cancer, adenocarcinoma, and thymic cancer, preferably brain cancer, but is not limited thereto. The pharmaceutical composition of the present invention may further comprise a conventional anticancer agent such as a chemical anticancer agent, immunotherapeutic agent, or immune checkpoint inhibitor, or may be used in combination with such an agent. The chemical anticancer agent may be at least one selected from the group consisting of: nitrogen mustard, imatinib, oxaliplatin, rituximab, erlotinib, neratinib, lapatinib, gefitinib, vandetanib, nilotinib, semaxanib, bosutinib, axitinib, cediranib, lestaurtinib, trastuzumab, gefitinib, bortezomib, sunitinib, carboplatin, sorafenib, bevacizumab, cisplatin, cetuximab, viscum album, asparaginase, tretinoin, hydroxycarbamide, dasatinib, estramustine, gemtuzumab ozogamicin, ibritumomab tiuxetan, heptaplatin, methyl aminolevulinate, amsacrine, alemtuzumab, procarbazine, alprostadil, holmium nitrate chitosan, gemcitabine, doxifluridine, pemetrexed, tegafur, capecitabine, gimeracil, oteracil, azacitidine, methotrexate, uracil, cytarabine, fluorouracil, fludarabine, enocitabine, flutamide, capecitabine, decitabine, mercaptopurine, thioguanine, cladribine, carmofur, raltitrexed, docetaxel, paclitaxel, irinotecan, belotecan, topotecan, vinorelbine, etoposide, vinblastine, idarubicin, mitomycin, bleomycin, dactinomycin, pirarubicin, aclarubicin, peplomycin, temsirolimus, temozolomide, busulfan, ifosfamide, cyclophosphamide, melphalan, altretamine, dacarbazine, thiotepa, nimustine, chlorambucil, mitolactol, leucovorin, tretinoin, exemestane, aminoglutethimide, anagrelide, olaparib, vinorelbine tartrate, fadrozole, tamoxifen, toremifene, testolactone, anastrozole, letrozole, vorozole, bicalutamide, lomustine, 5-FU, vorinostat, entinostat, and carmustine. The immunotherapeutic agent may be at least one selected from the group consisting of pembrolizumab, ipilimumab, nivolumab, atezolizumab, and durvalumab, but is not limited thereto, and it will be apparent to those skilled in the art that any agent known to function as a chemical anticancer agent or immunotherapeutic agent may be used without limitation. Given the characteristics of the present invention, the pharmaceutical composition of the present invention exhibits an excellent effect in overcoming immune suppression, and thus, when used in combination with an immune checkpoint inhibitor, it is expected to produce a remarkable synergistic effect in cancer treatment. The immune checkpoint inhibitor may be at least one selected from the group consisting of PD-1 receptor inhibitors, PD-L1 ligand inhibitors, PD-L2 ligand inhibitors, CTLA-4 receptor inhibitors, LAG-3 receptor inhibitors, TIM-3 receptor inhibitors, BTLA receptor inhibitors, and KIR receptor inhibitors, and may specifically bind to at least one protein selected from the group consisting of PD-1, PD-L1, PD-L2, CTLA-4, LAG-3, TIM-3, BTLA, and KIR, but is not limited thereto, and it will be apparent to those skilled in the art that any agent known to function as an immune checkpoint inhibitor may be used without limitation.

In yet another embodiment of the present invention, there is provided a method for preventing or treating cancer, comprising administering to a subject the γδ T cell therapeutic produced by the above-described method for producing a γδ T cell therapeutic with increased proliferation rate or in vivo persistence.

In yet another embodiment of the present invention, there is provided the use of the γδ T cell therapeutic produced by the above-described method for the prevention or treatment of cancer.

### Advantageous Effects

The γδ T cells produced by the method of the present invention exhibit remarkably improved proliferation rate, in vivo persistence, and antitumor effect, and are expected to significantly improve the survival rate of cancer-bearing subjects when used alone or in combination with conventional cancer immunotherapies.

### DESCRIPTION OF DRAWINGS

FIG. 1 shows a comparison of cell proliferation rates between restimulated γδ T cells and control group according to one embodiment of the present invention.

FIG. 2 shows a comparison of cell survival rates between restimulated γδ T cells and control group according to one embodiment of the present invention.

FIG. 3 shows a comparison of cell persistence in co-culture between restimulated γδ T cells and control group according to one embodiment of the present invention.

FIG. 4 shows the changes in mRNA of restimulated γδ T cells confirmed by bulk RNA sequencing according to one embodiment of the present invention.

FIG. 5 shows the results of confirming the reduction in cell persistence in co-culture of restimulated Batf3 knockout γδ T cells according to one embodiment of the present invention.

FIG. 6 shows the results of confirming the reduction in in vivo persistence of restimulated Batf3 knockout γδ T cells according to one embodiment of the present invention.

FIG. 7 shows the results of confirming the change in mRNA expression level of Batf3 over time in restimulated γδ T cells according to one embodiment of the present invention.

FIG. 8 shows a plasmid map for producing Batf3-overexpressing γδ T cells according to one embodiment of the present invention.

FIG. 9 shows the results of confirming the sustained expression level of Batf3 in Batf3-overexpressing γδ T cells according to one embodiment of the present invention.

FIG. 10 shows the results of confirming the change in proliferation rate of Batf3-overexpressing γδ T cells during in vitro culture according to one embodiment of the present invention.

FIG. 11 shows the results of confirming the change in cell cycle rate of Batf3-overexpressing γδ T cells during in vitro culture according to one embodiment of the present invention.

FIG. 12 shows the results of confirming the increase in in vivo persistence of Batf3-overexpressing γδ T cell therapeutic according to one embodiment of the present invention.

FIG. 13 shows the results of confirming the antitumor cytokine production and degranulation ability of Batf3-overexpressing γδ T cells according to one embodiment of the present invention.

FIG. 14 shows the results of confirming the change in survival rate when Batf3-overexpressing γδ T cell therapeutic is administered to a brain tumor subject according to one embodiment of the present invention.

### MODE FOR INVENTION

Hereinafter, the present disclosure will be described in detail with reference to the following examples. However, the following examples are merely illustrative of the present disclosure, and the content of the present disclosure is not limited by the following examples.

### EXAMPLE

### Experimental Methods

### 1. Brain Tumor Mouse Model

In the in vivo persistence experiment of Batf3 knockout and Batf3-overexpressing γδ T cells, 8-week-old male C57BL/ 6 mice were used based on the date of tumor cell implantation, and 1×10\hat{ }5 mouse-derived brain tumor cells (GL261 cell line) were injected into the intracranial space of the mice. In the experiment to test the therapeutic efficacy of γδ T cells, 12-week-old male C57BL/ 6 mice were used based on the date of tumor cell implantation, and 1×10\hat{ }5 mouse-derived brain tumor cells (GL 261-mCherry cell line) were injected into the intracranial space of the mice. Survival rates were subsequently observed.

### 2. In Vitro Culture and Gene Transduction of γδ T Cell Therapeutic

After isolation of γδ T cells from the spleen of wild-type mice using magnetic cell separation technology, γδ T cells were stimulated with anti-γδ TCR antibody, anti-CD28 antibody, and Interleukin-2 (IL-2). For restimulation, the same stimulation was given five days after the first stimulation. To obtain a sufficient number of γδ T cells for confirming the therapeutic efficacy of Batf3-overexpressing γδ T cells, TCR β knockout mice were used, and the same stimulation procedure was applied. For gene transduction, retrovirus containing either a control plasmid or a Batf3-overexpression plasmid was used for γδ T cells without restimulation, and γδ T cells expressing the transduction selection marker Thy1.1 were sorted using magnetic cell separation technology. For 24 hours prior to flow cytometric analysis of in vitro-cultured γδ T cells, the cells were cultured in IL-2-deprived medium to better resemble the in vivo condition. In addition, γδ T cells were also cultured in IL-2-deprived medium during co-culture for the same purpose.

### 3. Administration of γδ T Cell Therapeutic

To confirm the in vivo persistence of the γδ T cell therapeutic, γδ T cells were intravenously injected 7 days after brain tumor cell implantation. After 5 days, brain tumors were harvested, and the ratio and number of injected γδ T cells within the tumor microenvironment were analyzed using flow cytometry.

Additionally, to confirm the therapeutic efficacy of the γδ T cell therapeutic, transduced γδ T cells were intravenously injected at 7, 13, or 19 days after tumor cell implantation. As a control group, DPBS was injected.

### Experimental Results

### 1. Confirmation of Increased In Vivo Persistence of γδ T Cell Therapeutic via Restimulation Culture Method

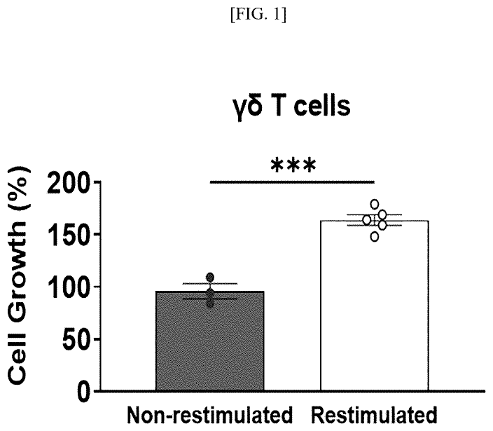

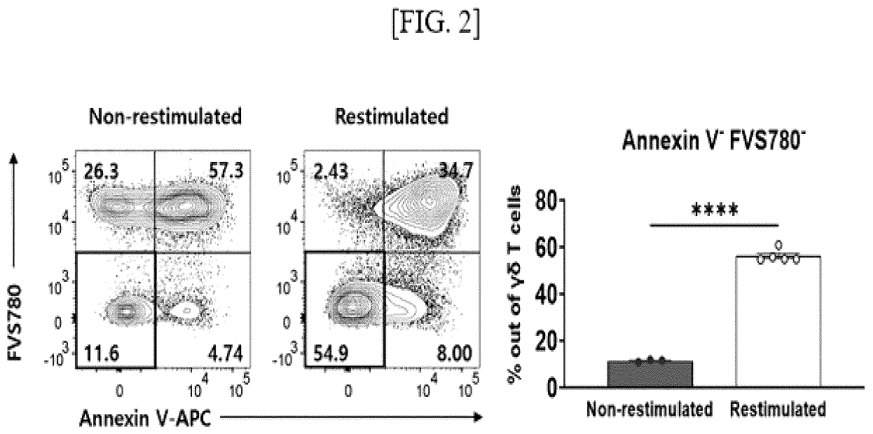

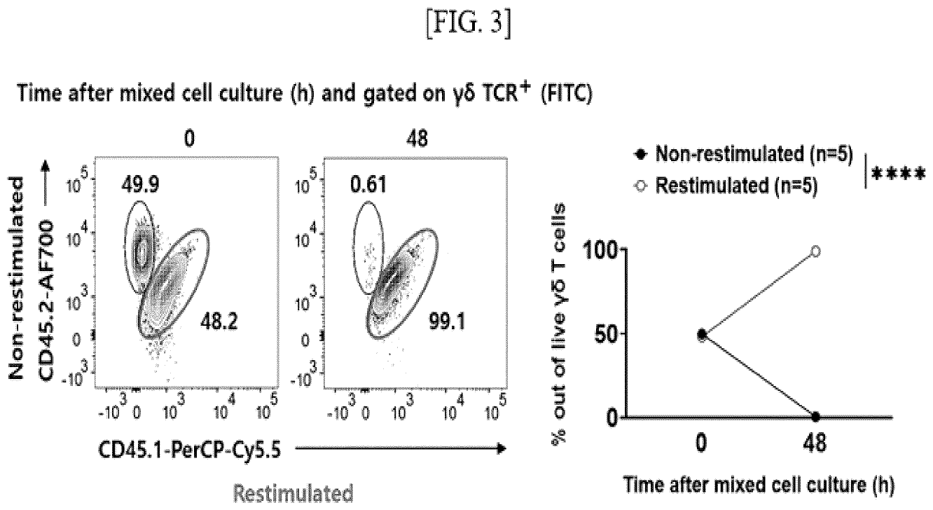

The restimulation culture method used in the present invention involves first stimulating the γδ TCR to promote ex vivo proliferation of γδ T cells, and restimulating 5 days later. As a result of checking the proliferation rate and apoptosis of γδ T cells after the restimulation culture, the restimulated γδ T cells showed significantly increased proliferation rate and cell viability compared to the non-restimulated control group (FIG. 1 and FIG. 2). Furthermore, when restimulated γδ T cells were co-cultured with non-restimulated γδ T cells at a 1:1 ratio, their persistence was significantly increased compared to the control group (FIG. 3). This indicates that the change in γδ T cells caused by restimulation is due to intrinsic cellular factors. These results demonstrate that the restimulation culture method can overcome one of the key limitations of γδ T cells, which is short persistence.

### 2. Confirmation Of Batf3 Upregulation By Restimulation and Subsequent Decrease Over Time

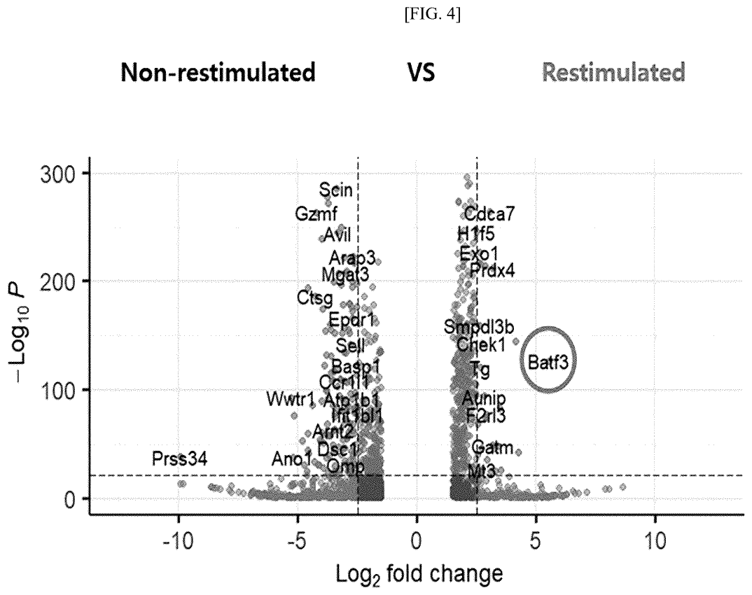

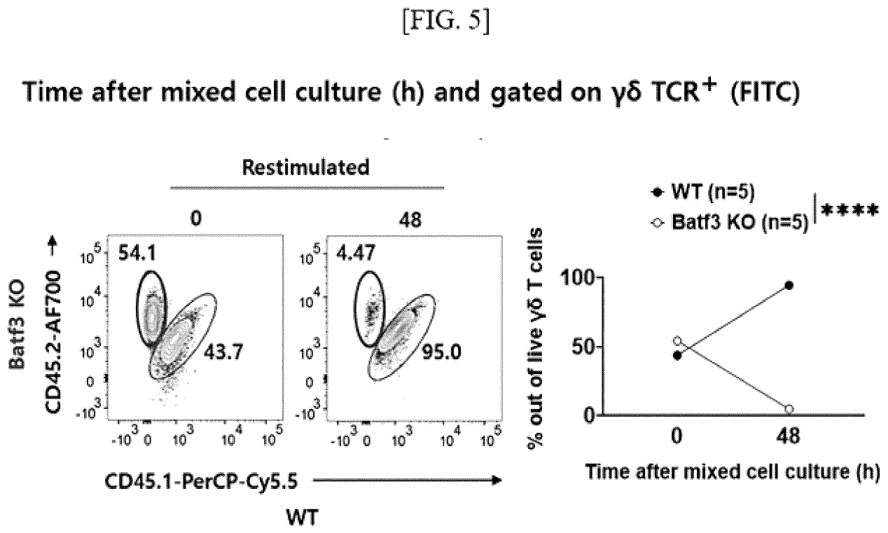

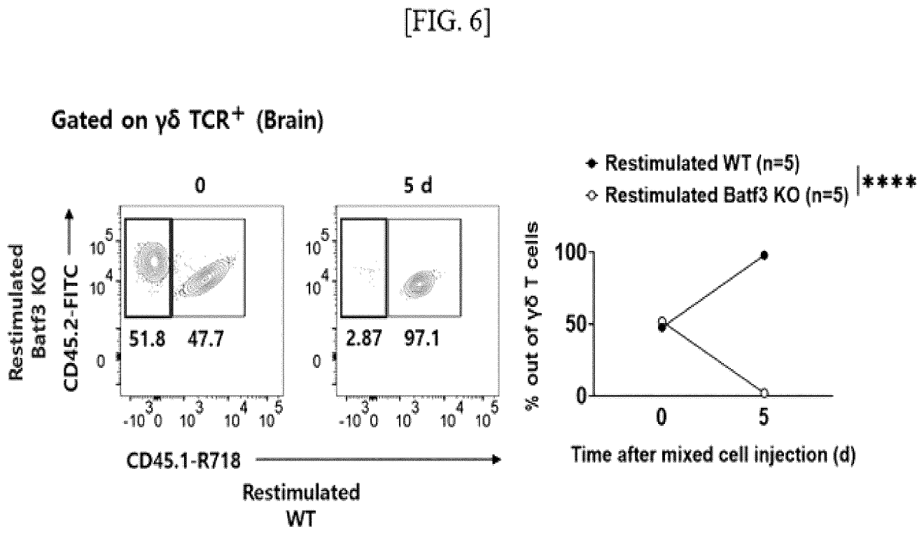

To explore the key mechanism by which restimulation affects γδ T cells, changes in mRNA of restimulated γδ T cells were confirmed through bulk RNA sequencing. As a result, Batf3 showed the most significant increase in expression among the genes statistically upregulated by restimulation compared to the control group (FIG. 4). To confirm whether Batf3 plays a key role in the increased persistence of γδ T cells due to restimulation, Batf3 knockout γδ T cells were co-cultured with wild-type (WT) γδ T cells after restimulation. As a result, the persistence of restimulated Batf3 knockout γδ T cells was markedly reduced compared to the control group (FIG. 5). In addition, when control and restimulated Batf3 knockout γδ T cells were co-transferred into a mouse model to analyze their in vivo kinetics, the persistence of Batf3 knockout γδ T cells in vivo was significantly reduced compared with the control group (FIG. 6). This indicates that increased Batf3 expression plays a key role in the in vivo persistence of restimulated γδ T cells.

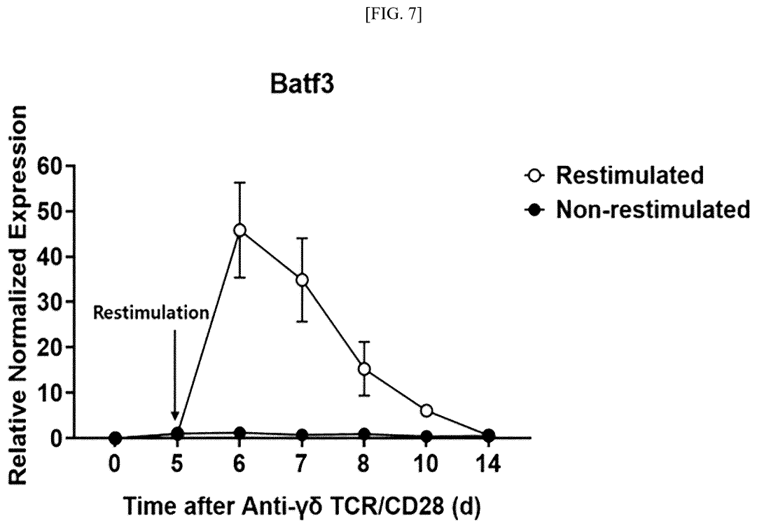

Additionally, real-time quantitative PCR confirmed that non-restimulated γδ T cells hardly expressed Batf3, and the Batf3 mRNA induced by restimulation decreased over time (FIG. 7). Thus, restimulation transiently increases Batf3 in γδ T cells.

These results suggest that long-term overexpression of Batf3 could enhance the in vivo persistence of γδ T cells, making them potentially useful as a therapeutic for brain tumors.

### 3. Production and Verification of Batf 3-Overexpressing γδ T Cells

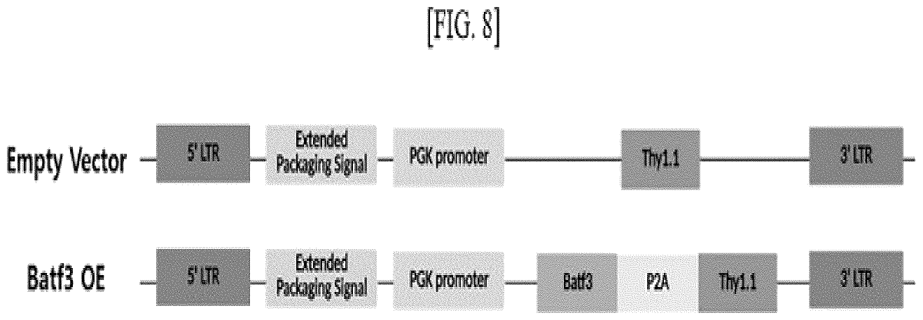

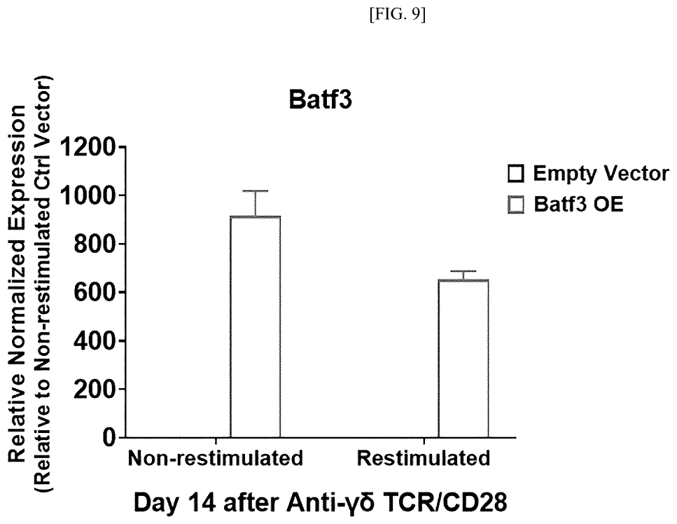

To produce Batf3-overexpressing γδ T cells, a plasmid was constructed containing the Batf3 gene and the selection marker Thy1.1 gene (FIG. 8). After transducing this plasmid into γδ T cells, the expression level of Batf3 was confirmed by qRT-PCR. The results showed that, unlike restimulated γδ T cells whose Batf3 expression almost completely disappeared over time, Batf3-overexpressing γδ T cells maintained a very high Batf3 expression level regardless of restimulation (FIG. 9).

### 4. Confirmation of Increased Proliferation and In Vivo Persistence of Non-Restimulated Batf3-Overexpressing γδ T Cells

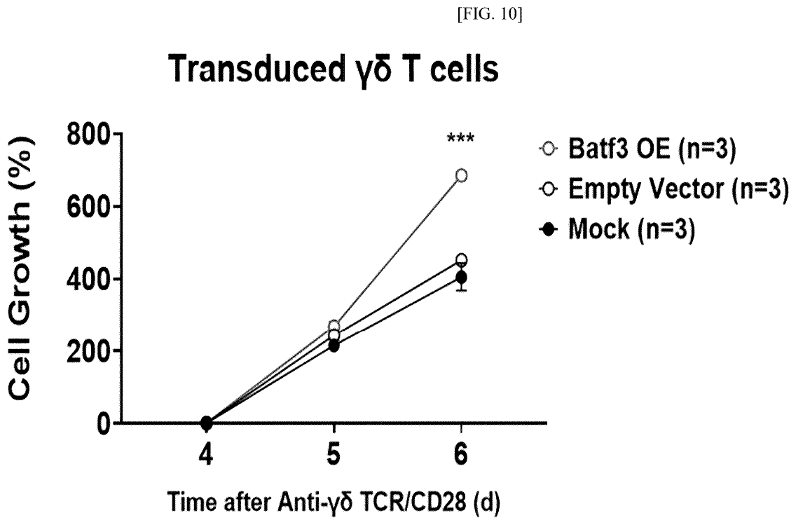

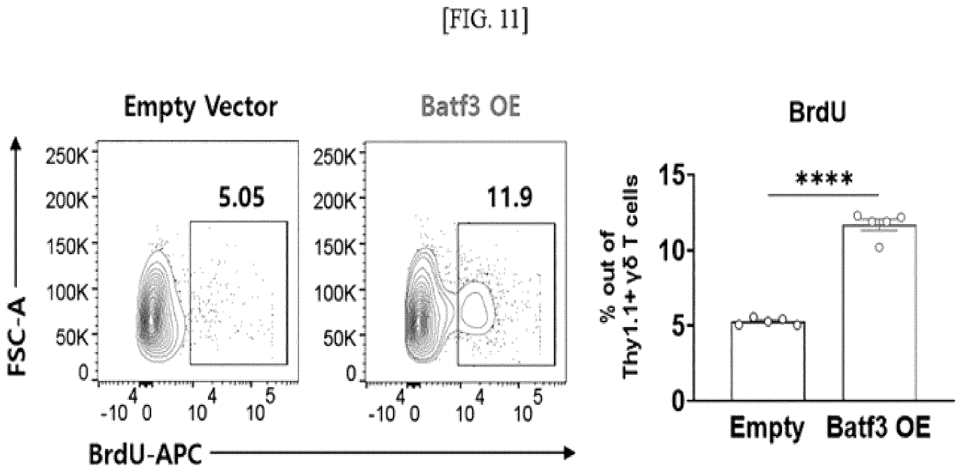

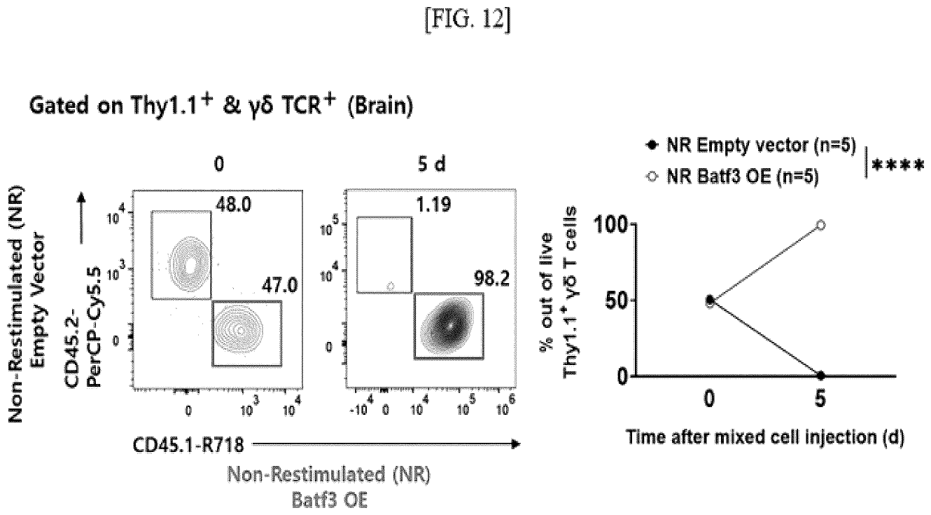

The proliferation rate and cell cycle progression of Batf3-overexpressing γδ T cells during in vitro culture were examined by flow cytometry. The results confirmed that the proliferation rate of Batf3-overexpressing γδ T cells was enhanced, and the proportion of BrdU+cells (S-phase markers) was increased, indicating a faster cell cycle progression (FIGS. 10 and 11). This correlates with the observed increase in proliferation rate. Furthermore, when Batf3-overexpressing γδ T cells and control γδ T cells were co-transferred into a mouse model for in vivo kinetic analysis, we observed that the in vivo persistence of Batf3-overexpressing γδ T cells was markedly increased compared with the control group (FIG. 12). This confirms that Batf3 overexpression improves the in vivo persistence of γδ T cell therapeutics.

### 5. Confirmation of Enhanced Antitumor Cytokine Production and Degranulation Capacity of Non-Restimulated Batf3-Overexpressing γδ T Cells

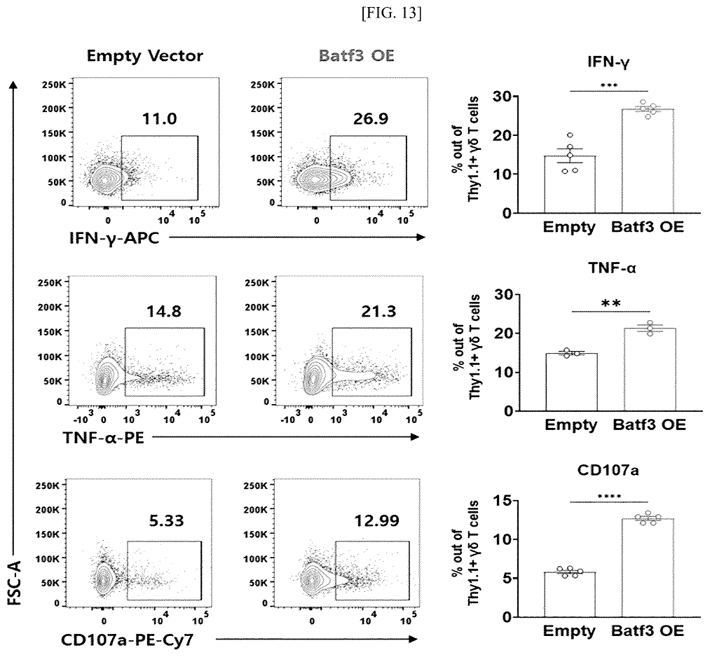

To examine the changes in antitumor immune response of Batf3-overexpressing γδ T cells, cytokine production and degranulation ability were analyzed by flow cytometry. The results showed that production of IFN-γ and TNF-α, which are representative antitumor cytokines, was significantly increased compared with the control group. In addition, expression of CD107a, a marker of degranulation capacity, was also increased (FIG. 13). These findings indicate that Batf3 overexpression enhances not only cytokine production but also secretion ability, thereby strengthening the antitumor immune response of γδ T cells.

[symbol]

6. Confirmation of Increased Survival in Mouse Brain Tumor Model Treated with Non-Restimulated Batf3-Overexpressing γδ T Cell Therapeutic

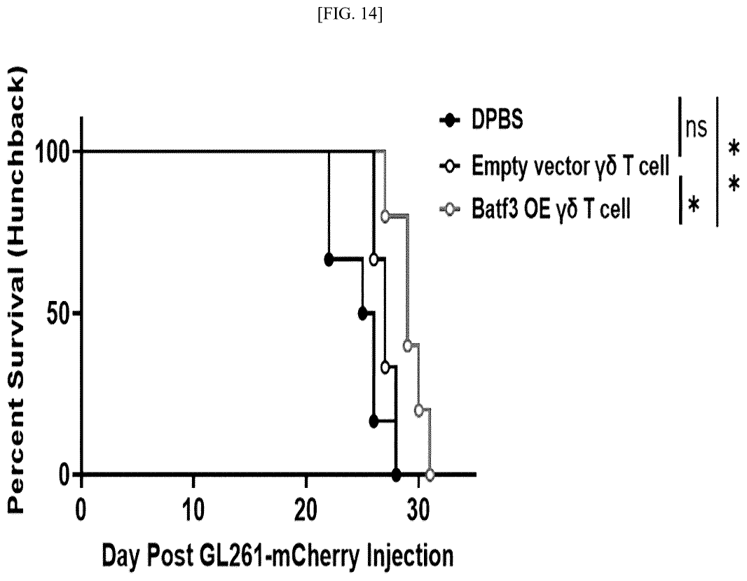

Finally, to confirm the efficacy of Batf3-overexpressing γδ T cells as a therapeutic for brain tumors, survival rate was compared after treatment. The results showed that treatment with Batf3-overexpressing γδ T cells significantly improved survival compared to the control group. By contrast, treatment with empty-vector γδ T cells (control) did not result in any survival benefit (FIG. 14). These findings demonstrate that Batf3-overexpressing γδ T cells, which overcome the limitations of conventional γδ T cell therapeutics, can be effectively used as a treatment for malignant brain tumors that have so far been very difficult to treat.

The research results of the present invention provide a new method to overcome the limitations of γδ T cell therapeutics, which until now have been impossible to apply clinically due to short in vivo persistence and decreased antitumor immune response. That is, by restimulating γδ TCR after primary stimulation during γδ T cell culture and/or by overexpressing Batf3, the proliferation rate and in vivo persistence can be significantly increased, and the immunosuppression in vivo can be overcome to enhance the antitumor immune response. The γδ T cell therapeutic cultured using the novel method of the present invention can be effectively applied to patients with glioblastoma, which is classified as an intractable disease.

Although the present disclosure has been described in detail with reference to the specific features, it will be apparent to those skilled in the art that this description is only of a preferred embodiment thereof, and does not limit the scope of the present disclosure. Thus, the substantial scope of the present disclosure will be defined by the appended claims and equivalents thereto.

1. A pharmaceutical composition for the prevention or treatment of cancer comprising γδ T cells with increased expression of Batf3 (Basic Leucine Zipper ATF-Like Transcription Factor 3) as an active ingredient.

2. The pharmaceutical composition according to claim 1, wherein the composition is used in combination with a chemical anticancer agent, an immunotherapeutic agent, or an immune checkpoint inhibitor, resulting in a remarkable synergistic effect.

3. A method for producing a γδ T cell therapeutic with increased proliferation rate or in vivo persistence, comprising stimulating a γδ TCR (T cell antigen receptor) of γδ T cells isolated from a subject.

4. The method according to claim 3, further comprising restimulating the γδ TCR of the γδ T cells or overexpressing Batf3.

5. The method according to claim 3, wherein the γδ TCR stimulation or restimulation is performed by treating with a composition comprising an anti-γδ TCR antibody.

6. The method according to claim 5, wherein the composition further comprises one or more selected from the group consisting of anti-CD28 antibody, Interleukin-2 (IL-2), Interleukin-5 (IL-5), Zoledronic acid, PHA, Transforming growth factor beta, Concanavalin A, and Amphotericin B.

7. The method according to claim 4, wherein the restimulation is performed 3 to 7 days after the primary stimulation of the γδ TCR of the γδ T cells.

8. The method according to claim 4, wherein the overexpression of Batf3 is performed 3 to 72 hours after the primary stimulation of the γδ TCR of the γδ T cells.

9. The method according to claim 4, wherein the overexpression of Batf3 is performed by transducing a vector comprising an PGK promoter (phosphoglycerate kinase 1 promoter) and Batf3 gene.

10. A method for improving the proliferation rate or in vivo persistence of γδ T cells, comprising stimulating a γδ TCR of γδ T cells isolated from a subject.

11. A γδ T cell therapeutic produced by the method according to claim 3.

12. The γδ T cell therapeutic according to claim 11, wherein the γδ T cell therapeutic is for cancer treatment.

13. The γδ T cell therapeutic according to claim 12, wherein the cancer is selected from the group consisting of brain cancer, breast cancer, cervical cancer, melanoma, lung cancer, bladder cancer, prostate cancer, leukemia, kidney cancer, liver cancer, colon cancer, pancreatic cancer, stomach cancer, gallbladder cancer, ovarian cancer, lymphoma, osteosarcoma, uterine cancer, oral cancer, bronchial cancer, nasopharyngeal cancer, laryngeal cancer, skin cancer, hematological cancer, thyroid cancer, parathyroid cancer, ureter cancer, adenocarcinoma, and thymus cancer.

14. A pharmaceutical composition for the prevention or treatment of cancer comprising the γδ T cell therapeutic according to claim 11 as an active ingredient.

15. The pharmaceutical composition according to claim 14, wherein the composition is used in combination with a chemical anticancer agent, an immunotherapeutic agent, or an immune checkpoint inhibitor.

16. The pharmaceutical composition according to claim 15, wherein the chemical anticancer agent is one or more selected from the group consisting of Nitrogen mustard, Imatinib, Oxaliplatin, Rituximab, Erlotinib, Neratinib, Lapatinib, Gefitinib, Vandetanib, Nilotinib, Semaxanib, Bosutinib, Axitinib, Cediranib, Lestaurtinib, Trastuzumab, Gefitinib, Bortezomib, Sunitinib, Carboplatin, Sorafenib, Bevacizumab, Cisplatin, Cetuximab, Viscum album, Asparaginase, Tretinoin, Hydroxycarbamide, Dasatinib, Estramustine, Gemtuzumab ozogamicin, Ibritumomab tiuxetan, Heptaplatin, Methyl aminolevulinate, Amsacrine, Alemtuzumab, Procarbazine, Alprostadil, Holmium nitrate chitosan, Gemcitabine, Doxifluridine, Pemetrexed, Tegafur, Capecitabine, Gimeracil, Oteracil, Azacitidine, Methotrexate, Uracil, Cytarabine, Fluorouracil, Fludarabine, Enocitabine, Flutamide, Capecitabine, Decitabine, Mercaptopurine, Thioguanine, Cladribine, Carmofur, Raltitrexed, Docetaxel, Paclitaxel, Irinotecan, Belotecan, Topotecan, Vinorelbine, Etoposide, Vinblastine, Idarubicin, Mitomycin, Bleomycin, Dactinomycin, Pirarubicin, Aclarubicin, Peplomycin, Temsirolimus, Temozolomide, Busulfan, Ifosfamide, Cyclophosphamide, Melphalan, Altretamine, Dacarbazine, Thiotepa, Nimustine, Chlorambucil, Mitolactol, Leucovorin, Tretinoin, Exemestane, Aminoglutethimide, Anagrelide, Olaparib, Navelbine, Fadrozole, Tamoxifen, Toremifene, Testolactone, Anastrozole, Letrozole, Vorozole, Bicalutamide, Lomustine, 5FU, Vorinostat, Entinostat, and Carmustine.

17. The pharmaceutical composition according to claim 15, wherein the immunotherapeutic agent is selected from the group consisting of Pembrolizumab, Ipilimumab, Nivolumab, Atezolizumab, and Durvalumab.

18. The pharmaceutical composition according to claim 15, wherein the immune checkpoint inhibitor is selected from the group consisting of PD-1 receptor inhibitor, PD-L1 ligand inhibitor, PD-L2 ligand inhibitor, CTLA-4 receptor inhibitor, LAG-3 receptor inhibitor, TIM-3 receptor inhibitor, BTLA receptor inhibitor, and KIR receptor inhibitor.

19. The pharmaceutical composition according to claim 15, wherein the immune checkpoint inhibitor specifically binds to at least one protein selected from the group consisting of PD-1, PD-L1, PD-L2, CTLA-4, LAG-3, TIM-3, BTLA, and KIR.

20. A method for preventing or treating cancer, comprising administering the γδ T cell therapeutic according to claim 11 to a subject.
